(19) United States
(12) Patent Application Publication
(10) Pub. No.: US 2019/0314435 A1
(43) Pub. Date: Oct. 17, 2019

(54) METHOD OF TREATING OR REDUCING THE SEVERITY OF DERMATOLOGICAL CONDITIONS

(71) Applicant: ANKAA, LLC, Chevy Chase, MD (US)

(72) Inventors: Adrian COPIZ, Washington, DC (US); Xiangnan SU, Chevy Chase, MD (US); Russell GRANDIS, Chevy Chase, MD (US)

(21) Appl. No.: 16/309,826

(22) PCT Filed: Aug. 15, 2017
(86) PCT No.: PCT/US17/46954

§ 371 Date: Dec. 13, 2018

Related U.S. Application Data
(60) Provisional application No. 62/379,897, filed on Aug. 26, 2016.

(51) Int. Cl. A61K 36/28; A61K 36/51; A61K 9/06; A61K 9/00; A61K 47/18; A61K 47/10; A61K 47/02; A61K 47/14; A61K 47/44

(52) U.S. Cl. CPC A61K 36/28; A61K 36/51; A61K 9/06; A61K 9/0014; A61K 47/44; A61K 47/10; A61K 47/02; A61K 47/14; A61K 47/183

(57) ABSTRACT

The present disclosure provides compositions and methods for treating dermatological conditions using a formulation comprising Swertia chirata and Bidens pilosa extracts.

### BACKGROUND

Swertia chirata is a blue flowering plant native to the Himalayas. It has been used topically and internally in Ayurvedic medicine as well as in traditional Tibetan folk medicine. (See, Kumar, V. and Van Staden J., Frontiers in Pharmacology, 6:1-14 (Article 308) (2016)). Swertia chirata extracts are commercially available from several sources.

Bidens pilosa is a species of flowering perennial herb in the aster family that is native to a variety of regions, including the Americas, Africa, and Pacific islands. The flower and its extracts have been used internally and topically to treat a wide range of ailments, with modern medical research on the plant focusing on its potential anti-cancer properties. (See, Bartolome, A. P., et al., Evidence Based Comp. and Alt. Med.,2013:1-51 (2013)). Bidens pilosa extracts are commercially available from several sources.

### BRIEF SUMMARY

It has now been surprisingly discovered that many dermatological conditions can be treated via the topical administration of an effective amount of a formulation comprising a combination of swertia chirata and bidens pilosa extracts, for a period of time sufficient to reduce the severity of the dermatological condition.

The present disclosure provides a formulation of swertia chirata and bidens pilosa extracts comprising amounts of swertia chirata and bidens pilosa extracts and a pharmaceutically acceptable carrier. In certain embodiments, the carrier can comprise at least one of a fat, a siloxane, an emollient, an emulsifier, an alcohol, a polyol, a polyolether, a penetration enhancer, silica, water, or a combination of any of the foregoing.

In certain embodiments, the at least one fat is selected from the group consisting of lard, butter, palm oil, shea butter, mango butter, kokum butter, cocoa butter, decanoic acid, undecanoic acid, erucic acid, tetradeconol, trideconal, lauryl alcohol, beneicosane, mono decane, octadecane, ercosane, elemi resin, levulinic acid, coconut oil, linseed oil, pumpkin seed oil, borage seed oil, dimethyl sebacate, adipic acid, polyethylene glycol, diethylene glycol, monotetradecyl ether, diethylene glycol, heptaethycine glycol monododecyl ether, palmitate esters, stearate esters, polycaprolactone-block-polytetrahydro-furan-block-poly[di(ethyleneglycol)adipate], hydrogenated oils, squalane, petroleum, solid paraffin, carnuba wax, bees wax, lanolin, trilaurin, stearic acid, palmitic acid, capric acid, myristic acid, lauric acid, tallow, whale blubber, and combinations thereof.

In certain embodiments, the siloxane is at least one cyclic siloxane. In certain embodiments, the at least one cyclic siloxane is selected from the group consisting of cyclotetrasiloxane, cyclopentasiloxane (cyclomethicone), cyclohexasiloxane, and combinations thereof.

In certain embodiments, the at least one emollient is selected from the group consisting of lanolin, mineral oil, paraffin, petrolatum, red petrolatum, white ointment, white petrolatum, yellow ointment, castor oil, cocoa butter, coconut oil, corn oil, cottonseed oil, olive oil, peanut oil, persic oil, sesame oil, cetyl esters wax, cold cream, hydrophilic ointment, rose water ointment, cetyl alcohol, glycerin, hydrophilic petrolatum, isopropyl myristate, myristyl alcohol, oleyl alcohol, shark liver oil, caprylic capric triglyceride, and combinations thereof.

In certain embodiments, the formulation further comprises at least one preservative.

In certain embodiments, the preservative is selected from the group consisting of quaternary ammonium compounds, halogenated phenols, sorbic acid, potassium sorbate, benzoic acid, sodium benzoate, disodium ethylenediaminetetraacetic acid (EDTA), phenoxyethanol, ethylhexylglycerin and combinations thereof.

In certain embodiments, the emulsifier is selected from the group consisting of cationic, anionic, and nonionic emulsifiers.

In certain embodiments, the formulation is a cream, serum, foam, gel, lotion, ointment, solution, or paste.

In certain embodiments, the formulation can further include a masking agent. In a particular embodiment the masking agent is ethylene brassylate.

The present disclosure also provides a method of treating a disease or disorder of the skin, comprising administering to a subject in need thereof, an effective amount of a topical formulation comprising swertia chirata and bidens pilosa extracts and a pharmaceutically acceptable carrier.

In certain embodiments, the disease or disorder of the skin is selected from the group consisting of fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, skin cancer, sunburn, dermatitis, rashes, impetigo, rhinophyma, perioral dermatitis, pseudofolliculitis barbae, erythema multiforme, erythema nodosum, granuloma annulare, alopecia, ichthyosis vulgaris, fungal infections, herpes simplex, keloids, milia, moluscum contagiosum, urticarial, vascular tumors and malformations, and combinations thereof.

In certain embodiments, the carrier comprises at least one of a fat, a siloxane, an emollient, an emulsifier, alcohol, polyol, polyolether, penetration enhancer, silica or a combination of any of the foregoing.

In certain embodiments, the at least one fat is selected from the group consisting of lard, butter, palm oil, shea butter, mango butter, kokum butter, cocoa butter, decanoic acid, undecanoic acid, erucic acid, tetradeconol, trideconal, lauryl alcohol, beneicosane, mono decane, octadecane, ercosane, elemi resin, levulinic acid, coconut oil, linseed oil, pumpkin seed oil, borage seed oil, dimethyl sebacate, adipic acid, polyethylene glycol, diethylene glycol, monotetradecyl ether, diethylene glycol, heptaethycine glycol monododecyl ether, palmitate esters, stearate esters, polycaprolactone-block-polytetrahydro-furan-block-poly[di(ethyleneglycol)adipate], hydrogenated oils, squalane, petroleum, solid paraffin, carnuba wax, bees wax, lanolin, trilaurin, stearic acid, palmitic acid, capric acid, myristic acid, lauric acid, tallow, whale blubber, and combinations thereof.

In certain embodiments, the at least one emollient is selected from the group consisting of lanolin, mineral oil, paraffin, petrolatum, red petrolatum, white ointment, white petrolatum, yellow ointment, castor oil, cocoa butter, coconut oil, corn oil, cottonseed oil, olive oil, peanut oil, persic oil, sesame oil, cetyl esters wax, cold cream, hydrophilic ointment, rose water ointment, cetyl alcohol, glycerin, hydrophilic petrolatum, isopropyl myristate, myristyl alcohol, oleyl alcohol, shark liver oil, and combinations thereof.

In certain embodiments, the emulsifier is selected from the group consisting of cationic, anionic, and nonionic emulsifiers.

In certain embodiments, the formulation further comprises at least one preservative.

In certain embodiments, the at least one preservative is selected from the group consisting of quaternary ammonium compounds, halogenated phenols, sorbic acid, potassium sorbate, benzoic acid, sodium benzoate, disodium ethylenediaminetetraacetic acid (EDTA), phenoxyethanol, ethylhexylglycerin and combinations thereof

In certain embodiments, the formulation is a cream, serum, foam, gel, lotion, ointment, solution, or paste.

### DETAILED DESCRIPTION

The articles “a,” “an,” and “the” are used herein to refer to one or to more than one (i.e., to at least one) of the grammatical object of the article. By way of example, “an element” means one element or more than one element.

The term “about” is used herein with numerical values to mean “within 10% of the stated value.” For example, “about 5 weight %” means from 4.5 weight % to 5.5 weight %.

The present disclosure provides a method of treating dermatological conditions comprising topically administering to a subject in need thereof an effective amount of a formulation comprising of a combination from about 0.005 to about 5.00 wt. % swertia chirata extract and from about 0.005 to about 5.00 wt. % bidens pilosa extract, for a period of time sufficient to reduce the severity of the dermatological condition. In particular embodiments, the formulation can comprise from about 0.005 to about 4.00 weight % Swertia chirata extract, from about 0.005 to about 3.00 weight % Swertia chirata extract, from about 0.005 to about 2.00 weight % Swertia chirata extract, from about 0.005 to about 1.00 weight % Swertia chirata extract, from about 0.005 to about 0.5 weight % Swertia chirata extract, from about 0.005 to about 0.25 weight % Swertia chirata extract, from about 0.005 to about 0.1 weight % Swertia chirata extract, or from about 0.005 to about 0.05 weight % Swertia chirata and from about 0.005 to about 4.00 weight % Bidens pilosa extract, from about 0.005 to about 3.00 weight % Bidens pilosa extract, from about 0.005 to about 2.00 weight % Bidens pilosa extract, from about 0.005 to about 1.00 weight % Bidens pilosa extract, from about 0.005 to about 0.5 weight % Bidens pilosa extract, from about 0.005 to about 0.25 weight % Bidens pilosa extract, from about 0.005 to about 0.1 weight % Bidens pilosa extract, or from about 0.005 to about 0.05 weight % Bidens pilosa extract.

In particular embodiments, the formulation can comprise about 0.005 weight % Swertia chirate extract, about 0.05 weight % Swertia chirate extract, about 0.1 weight % Swertia chirate extract, about 0.15 weight % Swertia chirate extract, about 0.25 weight % Swertia chirate extract, or about 0.5 weight % Swertia chirate extract and about 0.005 weight % Bidens pilosa extract, about 0.05 weight % Bidens pilosa extract, about 0.1 weight % Bidens pilosa extract, about 0.15 weight % Bidens pilosa extract, about 0.25 weight % Bidens pilosa extract, or about 0.5 weight % Bidens pilosa extract. Although extracts for use in the formulations described herein can be obtained from various commercial sources typically as part of a premixed composition (e.g., Lucas Meyer Cosmetics (Quebec, Calif.) (maltodextrin and Swertia chirate extract (SWT-7^{TM} H)) and Chemyunion (Morganville, N.J.) (Bidens pilosa extract, palm oil, cottonseed oil, and linseed oil (Revinage®)), in certain embodiments, the extracts can be prepared directly using standard extractive techniques known to those of ordinary skill in the art.

The formulations described herein can be substantially free of mercury, lead, and/or hormones. As used herein, the phrase “substantially free” with respect to mercury and lead means, not more than the quantities of these materials permitted by the FDA. For example, in certain embodiments, the formulations herein contain less than about 1 PPM mercury and less than about 20 PPM lead. With respect to hormones, the phrase “substantially free” means not more than about 100 PPM, not more than about 50 PPM, not more than about 10 PPM, not more than about 1 PPM, not more than about 0.1 PPM, not more than about 0.001 PPM, or not more than about 0.0001 PPM of any given hormone or combination of hormones.

In certain embodiments, the dermatological condition can be fine lines, wrinkles, dry skin, excessive pore size, skin dyschromia, reduced elasticity, unwanted hair, skin thinning, purpura, actinic keratosis, pruritus (itching), eczema, acne, rosacea, erythema (redness), telangiectasia (spider veins), skin cancer (including basal cell carcinoma, squamous cell carcinoma, and melanoma), sunburn, dermatitis, rashes, impetigo, rhinophyma, perioral dermatitis, pseudofolliculitis barbae (barber's itch), erythema multiforme (a hypersensitivity reaction), erythema nodosum, granuloma annulare, alopecia, ichthyosis vulgaris, fungal infections, herpes simplex, keloids, milia, moluscum contagiosum, urticarial (hives), vascular tumors and malformations, psoriasis, genital warts (condyloma acuminata), as well as combinations of the foregoing.

In one embodiment, the dermatological condition is dry skin. In another embodiment, the dermatological condition is wrinkles. In certain embodiments, the dermatological condition is a combination of dry skin and wrinkles.

While many of the dermatological conditions described herein can occur at any age, many are also age-related skin conditions that become more pronounced with age, exposure to harsh environments (including UV exposure, wind, cold, dry climate, etc.), or a combination of both. Exemplary age-related skin conditions include, but are not limited to, fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, and rhinophyma.

Without wishing to be bound by any particular theory, fine lines and wrinkles are believed to arise because of a breakdown of collagen and elastin in the skin caused and/or exacerbated by exposure to harmful UV radiation.

Dry skin is associated with itching, burning, and cracking of the epidermis. Dry skin can have many causative factors including, but not limited to, wind, extreme temperatures (both hot and cold), and air-conditioning, all of which cause the skin to lose moisture.

Although pore size is determined, to a certain extent, by genetics—pores can become larger with age or repeated sun exposure. Pores can also appear larger and more noticeable when clogged with dirt, oil and/or dead skin cells.

Skin dyschromias are discolorations (either lightening or darkening) of the epidermis. Although there are many known dyschromias, exemplary dyschromias suitable for treatment with the formulations described herein include post-inflammatory hyperpigmentation (PIH) and melasma.

Reduced elasticity in skin is often associated with changes in the connective tissue that reduce the skin's strength and elasticity. Reduced elasticity is especially pronounced after prolonged sun-exposure. Common features of reduced skin elasticity include the leathery, weather-beaten appearance common to those individuals who spend a large amount of time outdoors.

Purpura is the appearance of red or purple discolorations on the skin that do not blanch on applying pressure. The discolorations associated with purpura are caused by bleeding underneath the skin usually secondary to vasculitis or dietary deficiency of vitamin C (scurvy). They are also common in older individuals (senile purpura).

Actinic keratosis is a premalignant condition associated with photo-damaged skin. Actinic keratoses, also called AKs (they rarely appear alone) typically appear on sun-exposed areas such as the face, scalp, lips, and the back of the hands. AKs are often elevated, rough in texture, and resemble warts. Untreated AKs can advance to squamous cell carcinoma (SCC).

Eczema is inflammation of the skin, characterized by itchy, erythematous, vesicular, weeping, and crusting patches. Although the etiology of eczema is not well understood, it is believed to have an autoimmune component.

Rosacea is a chronic skin condition characterized by redness of the facial skin. Of the four known subtypes, the formulations described herein are suitable for treating erythematotelangiectatic rosacea, papulopustular rosacea, and phymatous rosacea.

Impetigo is a highly contagious skin disease common among school children. Impetigo usually appears as red sores on the face, especially around a child's nose and mouth. The sores burst and develop honey-colored crusts.

Rhinophyma is a large, bulbous, ruddy nose caused by granulomatous infiltration, commonly due to untreated phymatous rosacea.

Perioral dermatitis is skin disease characterized by multiple small papules, pustules, and vesicles which are localized in and around the perioral skin, nasolabial folds, or perioccular area.

Erythema nodosum (EN) is an inflammatory condition characterized by inflammation of fat cells under the skin, resulting in tender red nodules or lumps on the shins, buttocks, calves, ankles, thighs, and/or arms.

Granuloma annulare is a skin condition that most commonly consists of raised, reddish or skin-colored lesions that form ring patterns—usually on the backs of the forearms, hands, and/or feet. Sometimes the lesions can burn or itch. The lesions are caused by the clustering of T cells below the skin.

Ichthyosis vulgaris is a genetic skin disorder causing dry, scaly skin.

A keloid is the result of an overgrowth of granulation tissue (collagen type 3) at the site of a healed skin injury which is then slowly replaced by collagen type 1. Keloids are firm, rubbery lesions or shiny, fibrous nodules, and can vary in shape and color.

Milia are small white bumps or cysts on the skin almost always observed in newborn babies.

Moluscum contagiosum is a viral infection of the skin or mucous membrane. It is caused by a DNA poxvirus called the molluscum contagiosum virus (MCV).

In particular embodiments, the formulation can be a topically acceptable formulation, including, but not limited to an emulsion (such as a lotion, cream, shampoo, etc.), a serum, a wax, a gel, an oil, a foam, an ointment, a solution, or a paste. Emulsions can be oil in water emulsions or water in oil emulsions, many examples of which are known in the art. In a particular embodiment, the formulation can be a cream, a serum, a foam, a gel, a lotion, an ointment, a solution, or a paste.

In certain embodiments, the formulation can further include a masking agent. In a particular embodiment the masking agent is ethylene brassylate.

When the formulation is a water in oil emulsion, the formulation can comprise from about 1 to about 30 weight percent water. In other embodiments, the formulation can comprise from about 40 to about 80 weight percent water. In other embodiments, the formulation can comprise from about 60 to about 90 weight percent.

In some embodiments, the Swertia chirate and Bidens pilosa extracts can be completely suspended in the formulation. In other embodiments, the Swertia chirate and Bidens pilosa extracts can be completely soluble in the formulation. And in still other embodiments, a portion of the Swertia chirate and Bidens pilosa extracts in the formulation can be suspended while the remainder of the Swertia chirate and Bidens pilosa extracts can be solubilized. In some embodiments, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the Swertia chirate extract can be suspended in the formulation and about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the Bidens pilosa extract can be suspended in the formulation. In other embodiments, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the Swertia chirate extract can be solubilized in the formulation and about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the Bidens pilosa extract can be solubilized in the formulation.

In embodiments, wherein some amount of the Swertia chirate and Bidens pilosa extracts are suspended in the formulation, they can be suspended uniformly. That is, the suspended Swertia chirate and Bidens pilosa extracts will be dispersed evenly throughout the formulation such that separate aliquots of the formulation taken from different locations within the same batch will have substantially similar concentration of the suspended Swertia chirate and Bidens pilosa extracts.

In addition to Swertia chirate and Bidens pilosa extracts and water, the formulation suitable for use in the methods described herein can also include other ingredients commonly used in skin care and hair care products such as antimicrobials, anti-inflammatories, moisturizers, waxy alcohols, hydration agents, penetration enhancers, emulsifiers, natural or synthetic oils, solvents, fats, surfactants, detergents, gelling agents, emollients, antioxidants, fragrances, fillers, thickeners, waxes, odor absorbers, dyestuff, coloring agents, powders, viscosity-controlling agents, anesthetics, anti-itch agents, botanical extracts, conditioning agents, darkening or lightening agents, glitter, hair pigment additives, humectants, mica, minerals, polyphenols, silicones or silicone derivatives, sun blocks, vitamins, phytomedicinals, alcohols, polyols, polyolethers, penetration enhancers, and other compounds as listed in the International Cosmetic Ingredient Dictionary and Handbook, 13th Ed. (2009), the entirety of which is incorporated herein by reference. Although members of the various classes of compounds noted above can be mutually exclusive, in certain embodiments, a given ingredients can perform more than one function and can belong to more than one class.

In certain embodiments, the formulation can further include a hyaluronic acid derivative, such as a hyaluronin oligosaccharide. In particular embodiments, the hyaluronin oligosaccharides can have a molecular weight of from about 500 to less than about 50,000 daltons. In particular embodiments, the hyaluronin oligosaccharides can have a molecular weight of from about 500 to about 25,000 daltons. And in still further embodiments, the hyaluronin oligosaccharides can have a molecular weight of from about 500 to about 12,500 daltons. Exemplary hyaluronin oligosaccharides, and methods of making them, are disclosed in U.S. Published Application No. 2010/0098794, the entirety of which is incorporated herein by reference.

Exemplary vitamins suitable for use in the formulations described herein include, but are not limited to, panthenol, phytonadione, ascorbic acid, tocopheryl acetate, Niacinamide, and tetrahexyldecyl ascorbate.

Exemplary viscosity-controlling agents suitable for use in the formulations described herein include, but are not limited to, glycerin, hydroxyethyl cellulose, zinc stearate, polyacrylate crosspolymer-6 (commercially available from SEPPIC as SEPIMAX™ ZEN) and a composition of polyacrylate-13, polyisobutene, and polysorbate 20 (commercially available from SEPPIC as SEPIPLUS™ 400).

Exemplary conditioning agents suitable for use in the formulations described herein include, but are not limited to, acetyl glucosamine, acetyl carnitine, ethylhexyl palmitate, dipalmitoyl hydroxproline, methyl laurate, methyl linoleate, sodium pyrrolidone carboxylate, a composition of xylitylglucoside, anhydroxylitol, and xylitol (commercially available from SEPPIC as AQUAXYL), a composition of glycerin, laminaria saccharina extract, and arginine (commercially available from GELYMA as OCEA CARE®), bellis perennis flower extract (commercially available from CL Chemisches Laboratorium as BEILIDES), and a composition of caulerpa lentilfera extract and hydrolyzed rice protein (commercially available from GELYMA as OKINACEA EL).

Exemplary antimicrobials suitable for use in the formulations described herein include, but are not limited to, phenoxyethanol, ethylhexylglycerin, methylparaben, ethylparaben, propylparaben, butylparaben, and combinations thereof. In a particular embodiment, the formulation described herein can comprise a combination of methylparaben and ethylparaben. In other embodiments, the formulation can comprise propylparaben and/or phenoxyethanol. In still further embodiments, the formulation can comprise a combination of phenoxyethanol and ethylhexylglycerin.

Exemplary antioxidants suitable for use in the formulations described herein include, but are not limited to, butylatedhydroxytoulene (BHT) and butylatedhydroxyanisole (BHA). In one embodiment, the formulation can include an antioxidant in an amount from about 0.001 to about 3 weight %, or about 0.01 to about 1 weight %, or about 0.05 to about 1 weight % of each antioxidant or any combination thereof.

Exemplary waxy alcohols suitable for use in the formulations described herein include, but are not limited to, those alcohols having from fourteen carbon atoms to thirty carbon atoms, such as octadecanol, stearyl alcohol, n-heptadecanol, and nonadecyl alcohol. In particular embodiments, the formulation can comprise octadeanol. In other embodiments, the formulation can comprise stearyl alcohol. In still further embodiments, the formulation can comprise n-heptadecanol.

Exemplary fats suitable for use in the formulations described herein include, but are not limited to, lard, butter, palm oil, shea butter, mango butter, kokum butter, cocoa butter, decanoic acid, undecanoic acid, erucic acid, tetradeconol, trideconal, lauryl alcohol, beneicosane, mono decane, octadecane, ercosane, elemi resin, levulinic acid, coconut oil, dimethyl sebacate, adipic acid, polyethylene glycol, diethylene glycol, monotetradecyl ether, diethylene glycol, heptaethycine glycol monododecyl ether, palmitate esters, stearate esters, polycaprolactone-block-polytetrahydro-furan-block-poly[di(ethyleneglycol)adipate], hydrogenated oils, squalane, petroleum, solid paraffin, carnuba wax, bees wax, lanolin, trilaurin, stearic acid, palmitic acid, capric acid, myristic acid, lauric acid, tallow, whale blubber, and combinations thereof.

In a particular embodiment, the formulation described herein can comprise cocoa butter. In other embodiments, the formulation can comprise shea butter. In still further embodiments, the formulation can comprise a combination of shea butter and cocoa butter.

Exemplary emulsifiers suitable for use in the formulations described herein include, but are not limited to, cationic emulsifiers, anionic emulsifiers, nonionic emulsifiers, polysorbate 60, polysorbate 20, a composition of arachidyl alcohol, behenyl alcohol, and arachidyl glucoside (commercially available from SEPPIC as MONTANOV™ 202), glyceryl stearate, glyceryl monooleate, PEG stearates (such as, but not limited to, PEG-100 stearate, PEG-200 stearate, PEG-300 stearate, etc.), sorbitan sesquioleate, sorbitan olivate, sorbitan stearate, lecithin, undeceth-3, PEG-20 methyl glucose sesquistearate, trideceth-3, trideceth-12, laureth-9, behenoyl stearic acid, oleth-2, oleth-20, sorbitan laurate, sorbitan palmitate, sorbitan oleate, sorbitan trioleate, steareth-2, steareth-20, steareth-21, laureth-23, C11-15-Pareth-15, PPG-24-buteth-27, Avena sativa (oat) peptides, high molecular weight polymers of ethylene oxide and propylene oxide, PPG-5-ceteth-10 phosphate, oleth-5 phosphate, dioleyl phosphate, oleth-3 phosphate, oleth-10 phosphate, lauryl phosphate, trideceth-3 phosphate, trideceth-6 phosphate, deceth-6 phosphate, trilaureth-4 phosphate, C20-22 alkyl phosphate, C20-22 Alcohols, polyglyceryl-10 decaoleate, polyglyceryl-3 oleate, PEG/PPG-20/6 dimethicone, bis-PEG/PPG-20/20 dimethicone, bis-PEG/PPG-16/16 PEG/PPG-16/16 dimethicone, bis-PEG/PPG-20/5 PEG/PPG-20/5 dimethicone, methoxy PEG/PPG-25/4 dimethicone, bis-PEG/PPG-14/14 dimethicone, PEG-11 methyl ether dimethicone, PEG/PPG-20/22 butyl ether dimethicone, lauryl PEG-9 polydimethylsiloxyethyl dimethicone, PEG-10 dimethicone, polyglyceryl-3 disiloxane dimethicone, lauryl polyglyceryl-3 polydimethylsiloxyethyl dimethicone, lauryl PEG-8 dimethicone, sodium stearate, sucrose laurate, sucrose myristate, sucrose stearate, methyl glucose sesquistearate, and combinations of the foregoing. In some embodiments, the formulation can include an emulsifier in an amount from about 1 to about 15 weight %, and in other embodiments, the formulation can include an emulsifier in an amount of from about 1 to about 10 weight %, or from about 1 to about 5 weight %. If more than one emulsifier is used, the formulation can include from about 1 to about 5 weight % or from about 1.5 to about 3 weight % of each emulsifier.

In some embodiments, the emulsifier can be a PEG-stearate. In certain embodiments, the PEG-stearate can be PEG-100 stearate. In still other embodiments, the emulsifier can be a mixture of PEG-100 stearate and second emulsifier. In certain embodiments, the second emulsifier can be Avena sativa (oat) peptides. Without wishing to be bound by any particular theory, it is believed that oat peptide enhances skin's elasticity, promotes skin metabolism, and helps skin retain its moisture. Oat peptide is available from a variety of companies, including, for example, Shenyang Jihua Material Ltd. (China).

Exemplary natural and synthetic oils suitable for use in the formulations described herein include, but are not limited to, liquid paraffin, jojoba oil, grape seed oil, coconut oil, olive oil, castor oil, cottonseed oil, wheat germ oil, sunflower oil, safflower oil, carrot seed oil, and combinations thereof. In particular embodiments, the formulation can comprise jojoba oil. In other embodiments, the formulation can comprise grape seed oil. In certain embodiments, the formulation can comprise liquid paraffin in combination with at least a second oil. In particular embodiments, the second oil can be jojoba oil.

Exemplary emollients suitable for use in the formulations described herein include, but are not limited to, Isocetyl Palmitate (WAGLINOL 24416), Isocetyl Stearate (LASEMUL 244), Isodecyl Oleate (WEICHOL 158), Isononyl Isononanoate (WAGLINOL 1449), Isononyl Isononanoate (WAGLINOL 1449 NF), Isopropyl Isostearate (SOLDOC 60), Isopropyl Myristate (WAGLINOL 6014), Isopropyl Myristate/Isopropyl Palmitate (WAGLINOL 6014/16), Isopropyl Palmitate (WAGLINOL 6016), Isopropyl Stearate (LASEMUL 60), Isostearyl Isostearate (SOLDOC 272), Isotridecyl Isononanoate (WAGLINOL ITD 9), Myristyl Miristate (WAGLINOL 21414), Neopentyl Glycol Diethylhexanoate (WAGLINOL 2/1048), Neopentyl Glycol Diheptanoate (WAGLINOL 2/10407), Octyldodecyl Myristate (WAGLINOL 30014), lanolin, mineral oil, paraffin, petrolatum, red petrolatum, white ointment, white petrolatum, yellow ointment, castor oil, cocoa butter, coconut oil, corn oil, cottonseed oil, olive oil, peanut oil, persic oil, sesame oil, cetyl esters wax, cold cream, hydrophilic ointment, rose water ointment, cetyl alcohol, glycerin, hydrophilic petrolatum, isopropyl myristate, myristyl alcohol, oleyl alcohol, shark liver oil, caprylic capric triglyceride, and combinations of the foregoing. In a particular embodiment, the emollient is Isononyl Isononanoate (WAGLINOL 1449 NF). In one embodiment, the formulation can include an emollient, or a combination of emollients, in an amount from about 8 to about 30 weight %.

Exemplary humectants suitable for use in the formulations described herein include, but are not limited to, glycerol, sorbitol, alkylene glycols (e.g., propylene glycol, dipropylene glycol, polypropylene glycol, polyethylene glycol, hexylene glycol, butylene glycol, 1,3-butylene glycol, etc.), C₁₋₃alkoxylated glucose derivatives, glyceryl triacetate, hexanetriol, vinyl alcohol, xylitylglucoside, anhydroxylitol, xylitol, maltitol, polydextrose, alkoxylated glycerin (like ethoxylated glycerin), quillaia, urea, aloe vera gel, MP Diol (also known as 2-methyl-1,3-propane diol), alpha hydroxy acids (e.g., lactic acid), honey and combinations thereof. In particular embodiments, the formulation herein can comprise glycerol, sorbitol, propylene glycol, hexylene glycol, xylitylglucoside, anhydroxylitol, xylitol or a combination thereof.

Exemplary anti-inflammatories suitable for use in the formulations described herein include, but are not limited to, botanically-derived compounds, such as allantoin, witch hazel, aloe vera, chamomile, thyme extract, echinacea, purslane extract, or combinations thereof In particular embodiments, the formulation described herein can comprise allantoin.

Exemplary silicones or silicone derivatives suitable for use in the formulations described herein can be siloxanes. In some embodiments, the siloxanes can be cyclic siloxanes. In certain embodiments, the cyclic siloxanes can be cyclotetrasiloxane, cyclopentasiloxane (cyclomethicone), cyclohexasiloxane, and combinations thereof. In some embodiments, the formulation described herein can include at least one cyclic siloxane. In other embodiments, the cyclic siloxane can be cyclomethicone or cyclotetrasiloxane.

The formulation described herein can have a pH in the range of about 5 to about 7.5, and in certain embodiments, about 5 to about 7, about 6 to about 7, from about 6.1 to about 6.8, or from about 6.4 to about 6.6. In order to achieve the desired pH range, and in certain embodiments, the formulation described herein can include one or more pharmaceutically acceptable pH adjusting agents. Suitable pharmaceutically acceptable pH adjusting agents are known to those of ordinary skill in the art and include any pharmaceutically acceptable acids and bases.

In certain embodiments, the formulation can include a pH adjusting agent in an amount sufficient to achieve a desired pH range, i.e. from about pH 5 to about pH 7.5. In other embodiments, the formulation can contain from about 0.01 to about 1 weight %, or from about 0.05 to about 0.5 weight %, or from about 0.06 to about 0.15 weight %, or from about 0.06 to about 0.11 weight %, or from about 0.06 to about 0.1 weight % of pH adjusting agent. In certain embodiments, the formulation described herein can be substantially free of any pH adjusting agents.

In certain embodiments, the formulation described herein can include one or more preservatives. Exemplary preservatives suitable for use in the formulations described herein include, but are not limited to, quaternary ammonium compounds, halogenated phenols, sorbic acid, potassium sorbate, benzoic acid, sodium benzoate, disodium ethylenediaminetetraacetic acid (EDTA), a composition of phenoxyethanol and ethylhexylglycerin (commercially available from Schülke & Matr GmbH as EUXYL® PE 9010), and combinations of the foregoing. In one embodiment, the formulation can include a preservative in an amount from about 0.01 to about 3 weight %, or about 0.05 to about 1 weight %, or about 0.05 to about 0.5 weight %.

In certain embodiments, the formulation described herein can include one or more pharmaceutically acceptable alcohols. Exemplary pharmaceutically acceptable alcohols suitable for use in the formulations described herein include, but are not limited to, C1-C7 alcohols, such as ethanol, n-propanol, isopropanol, phenol, benzyl alcohol, and combinations thereof.

In certain embodiments, the formulation described herein can include one or more polyols. Exemplary polyols suitable for use in the formulations described herein include, but are not limited to, glycerol, ethylene glycol, propylene glycol, phytantriol, and combinations of the foregoing.

In certain embodiments, the formulation described herein can include one or more polyolethers. Exemplary polyolethers suitable for use in the formulations described herein include, but are not limited to, polyethylene glycol, polypropylene glycol, poly(ethylene glycol) methyl ether, poly(ethylene glycol) dimethyl ether, poly(ethylene glycol) ethyl ether, poly(ethylene glycol) diethyl ether, poly(propylene glycol) methyl ether, poly(propylene glycol) dimethyl ether, poly(propylene glycol) ethyl ether, poly(propylene glycol) diethyl ether, and combinations thereof.

In certain embodiments, the formulation described herein can include one or more penetration enhancers. Exemplary penetration enhancers suitable for use in the formulations described herein include, but are not limited to, fatty alcohols, fatty acids, lecithins, phospholipids, amines, amides, cyclodextrins, Brij, tweens, spas, pluronics, N-methyl pyrrolidone, ascorbate, sodium hyaluronate, dimethyl sulfoxide, ethanol, acetone, glycols, glycerol, squalene, tween 20 (polysorbate 20), azone, dodecyl pyrrolidone. dimethyl lauramide, benzyl alcohol, and combinations of the foregoing.

In certain embodiments, the formulation described herein can be a cream or a serum. In one embodiment, the formulation is a cream. In certain embodiments, the cream formulation can comprise a swertia chirata extract, a bidens pilosa extract, water, one or more preservatives, one or more viscosity-controlling agents, one or more emollients, one or more vitamins, one or more carriers, one or more natural or synthetic oils, one or more fats, one or more conditioning agents, and one or more emulsifiers.

In certain embodiments, the cream formulation further comprises a masking agent.

In certain embodiments, the masking agent is ethylene brassylate.

In certain embodiments of the cream formulation, the preservative is selected from the group consisting of quaternary ammonium compounds, halogenated phenols, sorbic acid, potassium sorbate, benzoic acid, sodium benzoate, disodium ethylenediaminetetraacetic acid (EDTA), phenoxyethanol, ethylhexylglycerin, and combinations thereof.

In certain embodiments of the cream formulation, the viscosity-controlling agent is selected from the group consisting of glycerin, polyacrylate crosspolymer-6 and a composition of polyacrylate-13, polyisobutene, and polysorbate 20, and a combination thereof.

In certain embodiments of the cream formulation, the emollient is selected from the group consisting of glycerin, caprylic capric triglyceride, cottonseed oil, and a combination thereof.

In certain embodiments of the cream formulation, the vitamin is selected from the group consisting of tocopheryl acetate, niacinamide, tetrahexyldecyl ascorbate, and a combination thereof.

In certain embodiments of the cream formulation, the carrier is selected from the group consisting of maltodextrin, silica, and a combination thereof.

In certain embodiments of the cream formulation, the natural or synthetic oil is selected from the group consisting of jojoba oil, grape seed oil, sunflower seed oil, cottonseed oil, and a combination thereof.

In certain embodiments of the cream formulation, the fat is selected from the group consisting of mango butter, palm oil, linseed oil, pumpkin seed oil, borage seed oil, and a combination thereof.

In certain embodiments of the cream formulation, the conditioning agent is selected from the group consisting of acetyl glucosamine, acetyl carnitine, ethylhexyl palmitate, dipalmitoyl hydroxproline, sodium pyrrolidone carboxylate, a composition of xylitylglucoside, anhydroxylitol, and xylitol, a composition of glycerin, laminaria saccharina extract, and arginine, and a composition of caulerpa lentilfera extract and hydrolyzed rice protein, and a combination thereof.

In certain embodiments of the cream formulation, the emulsifier is selected from the group consisting of cationic emulsifiers, anionic emulsifiers, nonionic emulsifiers, polysorbate 60, polysorbate 20, a composition of arachidyl alcohol, behenyl alcohol, and arachidyl glucoside, and a combination thereof.

In other embodiments, the formulation can be a serum. In certain embodiments, the serum formulation comprises a swertia chirata extract, a bidens pilosa extract, water, one or more preservatives, one or more viscosity-controlling agents, one or more emollients, one or more vitamins, one or more carriers, one or more natural or synthetic oils, one or more fats, one or more conditioning agents, and one or more emulsifiers.

In certain embodiments of the serum formulation, the preservative is selected from the group consisting of quaternary ammonium compounds, halogenated phenols, sorbic acid, potassium sorbate, benzoic acid, sodium benzoate, disodium ethylenediaminetetraacetic acid (EDTA), phenoxyethanol, ethylhexylglycerin, and combinations thereof.

In certain embodiments of the serum formulation, the viscosity-controlling agent is selected from the group consisting of glycerin, polyacrylate crosspolymer-6 and a composition of polyacrylate-13, polyisobutene, and polysorbate 20, and a combination thereof.

In certain embodiments of the serum formulation, the emollient is selected from the group consisting of glycerin, caprylic capric triglyceride, cottonseed oil, and a combination thereof.

In certain embodiments of the serum formulation, the vitamin is selected from the group consisting of tocopheryl acetate, Niacinamide, tetrahexyldecyl ascorbate, and a combination thereof.

In certain embodiments of the serum formulation, the carrier is selected from the group consisting of maltodextrin, silica, and a combination thereof.

In certain embodiments of the serum formulation, the natural or synthetic oil is selected from the group consisting of jojoba oil, grape seed oil, pumpkin seed oil, cottonseed oil, and a combination thereof.

In certain embodiments of the serum formulation, the fat is selected from the group consisting of mango butter, palm oil, linseed oil, pumpkin seed oil, borage seed oil, and a combination thereof.

In certain embodiments of the serum formulation, the conditioning agent is selected from the group consisting of acetyl glucosamine, acetyl carnitine, ethylhexyl palmitate, dipalmitoyl hydroxproline, sodium pyrrolidone carboxylate, a composition of xylitylglucoside, anhydroxylitol, and xylitol, a composition of glycerin, laminaria saccharina extract, and arginine, bellis perennis flower extract, and a composition of caulerpa lentilfera extract and hydrolyzed rice protein, and a combination thereof.

In certain embodiments of the serum formulation, the emulsifier is selected from the group consisting of cationic emulsifiers, anionic emulsifiers, nonionic emulsifiers, polysorbate 60, polysorbate 20, a composition of arachidyl alcohol, behenyl alcohol, and arachidyl glucoside, and a combination thereof.

### Methods of Treatment

Although the period of time sufficient to reduce the severity of the dermatological conditions discussed herein will vary depending upon the patient, the dermatological condition itself, and the concentration of the Swertia chirate and Bidens pilosa extracts in the formulation topically administered to the subject, in certain embodiments, the period of time sufficient to reduce the severity of the dermatological condition can be for example, about 52 weeks, about 36 weeks, about 26 weeks, about 18 weeks, about 16 weeks, about 15 weeks, about 14 weeks, about 13 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, or about 1 week.

The formulation described herein can be applied, for example, once daily, twice daily, three times daily, four time daily, or five times daily, for any of the periods of time noted above. In alternative embodiments, the formulation described herein can be applied once, twice, three time, four times, or five times a day every other day, every third day, every fourth day, every fifth day, every sixth day, or once a week for the periods of time noted above. For example, and without limitation, the formulation can be applied once a day every day, once a day every other day, twice a day every third day, once a day one time a week, etc.

The formulation described herein can be applied to any body surface, including, but not limited to, a facial surface, the scalp, neck, ears, shoulders, chest (including breasts and/or the décolletage), arms, hands, legs, stomach, buttocks, groin, back, feet, and combinations thereof. In particular embodiments, the facial surface can be the forehead, a perioral surface, a chin surface, a periorbital surface, a nasal surface, a cheek skin surface, or a combination thereof. A given body surface can be afflicted with one or more of the dermatological conditions described herein and more than one body surface can be treated at a time.

Effectiveness of the formulation for reducing the severity of the dermatological conditions can be measured using, for example, expert visual grading of high-resolution digital images taken at baseline (i.e., prior to treatment) and at other predetermined time points using the Rapid Evaluation of Anti-aging Leads (“REAL” 3.0) system. The REAL system and its use are described in “A randomized, controlled comparative study of the wrinkle reduction benefits.” J. J. J. Fu et al., British Journal of Dermatology, 162:647-654 (2010), the entirety of which is incorporated herein by reference.

According to the described method, three trained expert graders independently assess changes in the appearance of a given skin surface by comparing identified baseline and post-treatment images at given time points side-by-side using a ±eight-point ordinal scale. The expert graders and other assessors are blinded to the treatments. Although Fu et al used 8 and 24 weeks as the time points for comparison, other time points can be used as appropriate for a given dermatological condition. Similarly, although Fu et al, describes the use of expert graders, grading can be performed by computer.

### Preparation of the Formulations

The formulations described herein can be prepared according to known procedures. In a particular embodiment, a formulation described herein can be prepared by dissolving Swertia chirate and Bidens pilosa extracts in stearyl alcohol or other appropriate solvent to give a solution. A base formulation, comprising a mixture of the various elements disclosed herein, can then be prepared at a temperature in the range of from about 40° C. to about 90° C., and in certain embodiments, at about 70° C., at about 75° C. or at about 80° C. The Swertia chirate and Bidens pilosa solution can then be carefully added to the base formulation with mixing to give the formulation described herein.

The formulations described herein can be prepared using extracts from any and all parts of the Swertia chirate and Bidens pilosa plants. Exemplary parts of the Swertia chirata and Bidens pilosa plants include, but are not limited to, the roots, the leaves, the stem, the flowers, and the seeds, any of which can optionally be dried and/or powdered, and combinations of the foregoing. In other embodiments, whole plant extract, which can be dried and/or powdered, can be used. In one embodiment, a formulation can be prepared using extracts from the roots, which can optionally be dried and/or powdered. In another embodiment, a formulation can be prepared using extracts from the leaves, which can optionally be dried and/or powdered. In another embodiment, a formulation can be prepared using stem extracts, which can optionally be dried and/or powdered. In another embodiment, a formulation can be prepared using flower extracts, which can optionally be dried and/or powdered. In another embodiment, a formulation can be prepared using seed extracts, which can optionally be dried and/or powdered. In another embodiment, a formulation can be prepared using extracts from one of more of the Swertia chirate and Bidens pilosa plant parts, any of which can optionally be dried and/or powdered. Although a given formulation can include extracts from the same part or parts of both plants, in certain embodiments, the formulation can include mixed extracts, i.e. extracts from one part of one plant and a separate and distinct part of another plant. In still further embodiments, a given formulation can include extracts from more than one part of each plant, wherein the extracted plant parts can be the same or different.

In a particular embodiments, a given formulation can comprise leaf extract from Swertia chirate and whole plant extract from Bidens pilosa.

The phraseology or terminology herein is for the purpose of description and not of limitation. As such, the terminology and/or phraseology of the present specification should be interpreted by the skilled artisan in light of the teachings and guidance herein.

The breadth and scope of the present disclosure are not limited by any of the above-described exemplary embodiments, but instead are defined only in accordance with the following claims and their equivalents.

### EXAMPLES

### Example 1

### Cream Formulation Without Masking Agent

Cream Formulation without Masking Agent

| Component | Weight % |
|---|---|
| Water | 70.62 |
| Disodium EDTA | 0.10 |
| SepiMAX ™ Zen | 0.50 |
| Glycerin | 3.50 |
| Niacinamide | 0.50 |
| SWT-7 ™ H | 2.00 |
| Silica | 1.40 |
| Tetrahexyldecyl Ascorbate | 0.20 |
| Caprylic Capric Triglyceride | 2.63 |
| Grape seed Oil | 0.10 |
| Sunflower seed oil | 3.55 |
| Borage seed oil | 0.50 |
| Ethylhexyl Palmitate | 2.30 |
| MONTANOV ™ 202 | 1.50 |
| Revinage ® | 2.00 |
| Tocopheryl Acetate | 0.10 |
| Dipalmitoyl Hydroxyproline | 1.00 |
| Euxyl ® PE 9010 | 1.00 |
| Sepiplus ™ 400 | 1.40 |
| Sodium Pyrrolidone Carboxylate | 0.10 |
| Aquaxyl ™ | 3.00 |
| Ocea Care ® | 2.00 |
| Total | 100% |

### Example 2

### Cream Formulation With Masking Agent

Cream Formulation with Masking Agent

| Component | Weight % |
|---|---|
| Water | 69.62 |
| Disodium EDTA | 0.10 |
| SepiMAX ™ Zen | 0.50 |
| Glycerin | 3.50 |
| Niacinamide | 0.50 |
| SWT-7 ™ H | 2.00 |
| Silica | 1.40 |
| Tetrahexyldecyl Ascorbate | 0.20 |
| Caprylic Capric Triglyceride | 2.63 |
| Grape seed Oil | 0.10 |
| Sunflower seed oil | 3.55 |
| Borage seed oil | 0.50 |
| Ethylhexyl Palmitate | 2.30 |
| MONTANOV ™ 202 | 1.50 |
| Revinage ® | 2.00 |
| Tocopheryl Acetate | 0.10 |
| Dipalmitoyl Hydroxyproline | 1.00 |
| Euxyl ® PE 9010 | 1.00 |
| Sepiplus ™ 400 | 1.40 |
| Sodium Pyrrolidone Carboxylate | 0.10 |
| Aquaxyl ™ | 3.00 |
| Ocea Care ® | 2.00 |
| Ethylene Brassylate | 1.00 |
| Total | 100% |

### Example 3

### Serum Formulation

Serum Formulation

| Component | Weight % |
|---|---|
| Water | 76.10 |
| Disodium EDTA | 0.10 |
| SepiMAX ™ Zen | 0.70 |
| Glycerin | 3.50 |
| Niacinamide | 0.50 |
| SWT-7 ™ H | 2.00 |
| Silica | 0.50 |
| Tetrahexyldecyl Ascorbate | 0.50 |
| Polysorbate 60 | 0.30 |
| Mango Butter | 1.00 |
| pumpkin seed oil | 1.00 |
| Borage seed oil | 0.50 |
| jojoba oil | 1.00 |
| Revinage ® | 2.00 |
| Tocopheryl Acetate | 0.20 |
| Dipalmitoyl Hydroxyproline | 1.00 |
| Euxyl ® PE 9010 | 1.00 |
| Sodium Pyrrolidone Carboxylate | 0.10 |
| Aquaxyl ™ | 3.00 |
| Okinacea ® EL | 2.00 |
| Bellis Perennis Flower Extract | 3.00 |
| Total | 100% |

### Example 4

### Preparation of Cream Formulation Without Masking Agent

Process of Making a 100 g Batch of the
Cream Formulation without Masking Agent

| | | |
|---|---|---|
| Phase A: | Water | 70.62 |
| sprinkle Sepimax Zen ™ in water and EDTA | Disodium EDTA | 0.10 |
| while mixing | SEPIMAX ™ Zen | 0.50 |
| Phase B: | Glycerin | 3.50 |
| Add to phase A and warm to dissolved. | Niacinamide | 0.50 |
| Phase C: | SWT-7 ™ H | 2.00 |
| Sprinkle one at a time and mix until uniform. | silica | 1.40 |
| Heat to 75-80° C. | | |
| Phase D: | Tetrahexyldecyl | 0.20 |
| Premix in the main tank and heat to | Ascorbate | |
| 75-80° C. | Caprylic Capric | 2.63 |
| Add to phase A/B/C and homogenize lightly. | Triglyceride | |
| | Grapeseed Oil | 0.10 |
| | Sunflower seed oil | 3.55 |
| | Borage seed oil | 0.50 |
| | Ethylhexyl | 2.30 |
| | Palmitate | |
| | MONTANOV ™ | 1.50 |
| | 202 | |
| | Revinage ® | 2.00 |
| | Tocopheryl Acetate | 0.10 |
| | Dipalmitoyl | 1.00 |
| | Hydroxyproline | |
| Phase E: | EUXYL ® PE 9010 | 1.00 |
| Cool Batch to 70-73° C. and add to phase D | SEPIPLUS ™ 400 | 1.40 |
| with slight homogenization. | | |
| Phase F: | Sodium Pyrrolidone | 0.10 |
| Switch to sweep mixing and cool to | Carboxylate | |
| 40-43° C. and add one at a time to phase E, | Aquaxyl ™ | 3.00 |
| mixing until uniform. | Ocea Care ® | 2.00 |
| Cool to room temperature. | | |

### Example 5

### Preparation of Cream Formulation With Masking Agent

Process of Making a 100 g Batch of the
Cream Formulation with Masking Agent

| | | |
|---|---|---|
| Phase A: | Water | 70.62 |
| sprinkle Sepimax Zen ™ in water and EDTA | Disodium EDTA | 0.10 |
| while mixing | SEPIMAX ™ Zen | 0.50 |
| Phase B: | Glycerin | 3.50 |
| Add to phase A and warm to dissolved. | Niacinamide | 0.50 |
| Phase C: | SWT-7 ™ H | 2.00 |
| Sprinkle one at a time and mix until uniform. | silica | 1.40 |
| Heat to 75-80° C. | | |
| Phase D: | Tetrahexyldecyl | 0.20 |
| Premix in the main tank and heat to | Ascorbate | |
| 75-80° C. | Caprylic Capric | 2.63 |
| Add to phase A/B/C and homogenize lightly. | Triglyceride | |
| | Grapeseed Oil | 0.10 |
| | Sunflower seed oil | 3.55 |
| | Borage seed oil | 0.50 |
| | Ethylhexyl | 2.30 |
| | Palmitate | |
| | MONTANOV ™ | 1.50 |
| | 202 | |
| | Revinage ® | 2.00 |
| | Tocopheryl Acetate | 0.10 |
| | Dipalmitoyl | 1.00 |
| | Hydroxyproline | |
| Phase E: | EUXYL ® PE 9010 | 1.00 |
| Cool Batch to 70-73° C. and add to phase D | SEPIPLUS ™ 400 | 1.40 |
| with slight homogenization. | | |
| Phase F: | Sodium Pyrrolidone | 0.10 |
| Switch to sweep mixing and cool to | Carboxylate | |
| 40-43° C. and add one at a time to phase E, | Aquaxyl ™ | 3.00 |
| mixing until uniform. | Ocea Care ® | 2.00 |
| Cool to room temperature. | Ethylene Brassylate | 1.00 |

### Example 6

### Preparation of Serum Formulation

Process of Making a 100 g Batch of the Serum Formulation

| | | |
|---|---|---|
| Phase A: | Water | 76.10 |
| Sprinkle Sepimax Zen ™ in water and | Disodium EDTA | 0.10 |
| EDTA while mixing in side tank. | SEPIMAX ™ Zen | 0.70 |
| Phase B: Add the following to Phase A | Glycerin | 3.50 |
| Phase C: | Niacinamide | 0.50 |
| Sprinkle the following one at a time and mix | SWT-7 ™ H | 2.00 |
| until uniform. | Silica | 0.50 |
| Warm slightly. | | |
| Phase D: | Tetrahexyldecyl | 0.50 |
| Premix the following in main tank. | Ascorbate | |
| heat until dissolved and cool to room | Polysorbate 60 | 0.30 |
| temperature. | Mango Butter | 1.00 |
| | pumpkin seed oil | 1.00 |
| | Borage seed oil | 0.50 |
| | jojoba seed oil | 1.00 |
| | Revinage ® | 2.00 |
| | Tocopheryl | 0.20 |
| | Acetate | |
| | Dipalmitoyl | 1.00 |
| | Hydroxyproline | |
| Phase E: add to phase E | Euxyl PE 9010 | 1.00 |
| Phase F: | Sodium | 0.10 |
| Slowly add Phase A, B, C, and D to Phase | Pyrrolidone | |
| E and F homogenize lightly and add one at | Carboxylate | |
| a time | AQUAXYL ™ | 3.00 |
| | OKINACEA ® | 2.00 |
| | EL | |
| | Bellis Perennis | 3.00 |
| | Flower Extract | |

What is claimed is:

1. A formulation of swertia chirata and bidens pilosa extracts comprising an amount of swertia chirata and bidens pilosa extracts and a pharmaceutically acceptable carrier.

2. The formulation of claim 1, wherein the carrier comprises at least one of a fat, a siloxane, an emollient, an emulsifier, an alcohol, a polyol, a polyolether, a penetration enhancer, silica, water, or a combination of any of the foregoing.

3. The formulation of claim 2, wherein the at least one fat is selected from the group consisting of lard, butter, palm oil, shea butter, mango butter, kokum butter, cocoa butter, decanoic acid, undecanoic acid, erucic acid, tetradeconol, trideconal, lauryl alcohol, beneicosane, mono decane, octadecane, ercosane, elemi resin, levulinic acid, coconut oil, linseed oil, pumpkin seed oil, borage seed oil, dimethyl sebacate, adipic acid, polyethylene glycol, diethylene glycol, monotetradecyl ether, diethylene glycol, heptaethycine glycol monododecyl ether, palmitate esters, stearate esters, polycaprolactone-block-polytetrahydro-furan-block-poly[di(ethyleneglycol)adipate], hydrogenated oils, squalane, petroleum, solid paraffin, carnuba wax, bees wax, lanolin, trilaurin, stearic acid, palmitic acid, capric acid, myristic acid, lauric acid, tallow, whale blubber, and combinations thereof.

4. The formulation of claim 2, wherein the at least one emollient is selected from the group consisting of lanolin, mineral oil, paraffin, petrolatum, red petrolatum, white ointment, white petrolatum, yellow ointment, castor oil, cocoa butter, coconut oil, corn oil, cottonseed oil, olive oil, peanut oil, persic oil, sesame oil, cetyl esters wax, cold cream, hydrophilic ointment, rose water ointment, cetyl alcohol, glycerin, hydrophilic petrolatum, isopropyl myristate, myristyl alcohol, oleyl alcohol, shark liver oil, caprylic capric triglyceride, and combinations thereof.

5. The formulation of claim 2, further comprising at least one preservative.

6. The formulation of claim 2, further comprising a masking agent.

7. The formulation of claim 5, wherein the preservative is selected from the group consisting of quaternary ammonium compounds, halogenated phenols, sorbic acid, potassium sorbate, benzoic acid, sodium benzoate, disodium ethylenediaminetetraacetic acid (EDTA), phenoxyethanol, ethylhexylglycerin, and combinations thereof.

8. The formulation of claim 2, wherein the emulsifier is selected from the group consisting of cationic, anionic, and nonionic emulsifiers.

9. The formulation of claim 1 wherein the formulation is a cream, serum, foam, gel, lotion, ointment, solution, or paste.

10. A method of treating a disease or disorder of the skin, comprising administering to a subject in need thereof, an effective amount of a topical formulation comprising swertia chirata and bidens pilosa extracts and a pharmaceutically acceptable carrier.

11. The method of claim 10, wherein the disease or disorder of the skin is selected from the group consisting of fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, skin cancer, sunburn, dermatitis, rashes, impetigo, rhinophyma, perioral dermatitis, pseudofolliculitis barbae, erythema multiforme, erythema nodosum, granuloma annulare, alopecia, ichthyosis vulgaris, fungal infections, herpes simplex, keloids, milia, moluscum contagiosum, urticarial, vascular tumors and malformations, and combinations thereof.

12. The method of claim 11, wherein the carrier comprises at least one of a fat, a siloxane, an emollient, an emulsifier, alcohol, polyol, polyolether, penetration enhancer, silica or a combination of any of the foregoing.

13. The method of claim 12, wherein the at least one fat is selected from the group consisting of lard, butter, palm oil, shea butter, mango butter, kokum butter, cocoa butter, decanoic acid, undecanoic acid, erucic acid, tetradeconol, trideconal, lauryl alcohol, beneicosane, mono decane, octadecane, ercosane, elemi resin, levulinic acid, coconut oil, linseed oil, pumpkin seed oil, borage seed oil, dimethyl sebacate, adipic acid, polyethylene glycol, diethylene glycol, monotetradecyl ether, diethylene glycol, heptaethycine glycol monododecyl ether, palmitate esters, stearate esters, polycaprolactone-block-polytetrahydro-furan-block-poly[di(ethyleneglycol)adipate], hydrogenated oils, squalane, petroleum, solid paraffin, carnuba wax, bees wax, lanolin, trilaurin, stearic acid, palmitic acid, capric acid, myristic acid, lauric acid, tallow, whale blubber, and combinations thereof.

14. The method of claim 13, wherein the at least one emollient is selected from the group consisting of lanolin, mineral oil, paraffin, petrolatum, red petrolatum, white ointment, white petrolatum, yellow ointment, castor oil, cocoa butter, coconut oil, corn oil, cottonseed oil, olive oil, peanut oil, persic oil, sesame oil, cetyl esters wax, cold cream, hydrophilic ointment, rose water ointment, cetyl alcohol, glycerin, hydrophilic petrolatum, isopropyl myristate, myristyl alcohol, oleyl alcohol, shark liver oil, caprylic capric triglyceride, and combinations thereof.

15. The method of claim 12, wherein the emulsifier is selected from the group consisting of cationic, anionic, and nonionic emulsifiers.

16. The method of claim 12, further comprising at least one preservative.

17. The method of claim 12, further comprising a masking agent.

18. The method of claim 16, wherein the at least one preservative is selected from the group consisting of quaternary ammonium compounds, halogenated phenols, sorbic acid, potassium sorbate, benzoic acid, sodium benzoate, disodium ethylenediaminetetraacetic acid (EDTA), phenoxyethanol, ethylhexylglycerin, and combinations thereof.

19. The method of claim 10, wherein the formulation is a cream, serum foam, gel, lotion, ointment, solution, or paste.
